(19) United States
(12) Patent Application Publication
(10) Pub. No.: US 2025/0327110 A1
(43) Pub. Date: Oct. 23, 2025

(54) HIGH THROUGHPUT MULTIPLEXED SERUM BASED BACTERICIDAL ASSAYS (SBAs), AND KITS FOR USE THEREIN

(71) Applicant: SUNFIRE BIOTECHNOLOGIES, Birmingham, AL (US)

(72) Inventors: Robert L. Burton, Birmingham, AL (US); Moon H. Nahm, Birmingham, AL (US)

(21) Appl. No.: 19/185,485

(22) Filed: Apr. 22, 2025

Related U.S. Application Data
(60) Provisional application No. 63/637,582, filed on Apr. 23, 2024.

(51) Int. Cl. C12Q 1/18; G01N 33/68

(52) U.S. Cl. CPC C12Q 1/18; G01N 33/6854; G01N 2333/25

(57) ABSTRACT

The present disclosure provides multiplexed serum-based assays (SBA's) for detecting the efficacy and/or specificity of a putative vaccine to elicit protective or functional antibodies which elicit complement-mediated killing of 3 or more different gram-negative bacterial strains optionally of the same genus, wherein the SBA is conducted in multiplex format using (i) each of said 3 or more gram-negative bacterial strains which respectively are resistant to a different antibiotic relative to the other gram-negative bacterial strains and (ii) complement, e.g., baby rabbit complement.

### RELATED APPLICATIONS

The present application claims benefit of priority to U.S. Provisional Application No. 63/637,582 filed on Apr. 23, 2024, the contents of which are incorporated by reference in their entirety.

### GOVERNMENT FUNDING

This invention was made with government support under W81XWH-22-C-0016, and W81XWH-21-P-0022 awarded by the Defense Health Agency, Medical Research and Development Branch. The government has certain rights in the invention.

### FIELD OF THE INVENTION

The present invention provides multiplexed serum-based bactericidal assays (SBA's) for detecting the efficacy and/or specificity of a putative vaccine to elicit protective or neutralizing antibodies against 3 or more different gram-negative bacterial strains, optionally Shigella strains, and kits for use in such multiplexed SBA's.

### BACKGROUND

Infections caused by antibiotic resistant gram-negative bacteria are becoming increasingly prevalent and constitute a serious threat to public health worldwide because they are often difficult to treat and are associated with high morbidity and mortality rates. Gram-negative bacteria cause infections including pneumonia, bloodstream infections, wound or surgical site infections, and meningitis in healthcare settings. Moreover, gram-negative bacteria which are resistant to multiple drugs which are increasingly resistant to most available antibiotics are becoming more prevalent in health care settings. Based thereon there is a significant need for vaccines which provide for specific and durable protection against gram-negative bacteria, particularly vaccines which provide for protection in vulnerable subjects such as the elderly, infants and subjects with comorbidities are needed.

In view of the foregoing, assays for evaluating the efficacy of putative vaccines against Gram negative bacteria are needed, in particular high throughput vaccines which may be used to assess protection against different bacterial strains. An example thereof comprises multiplexed opsonophagocytic assays. The first reported use of such assays was for evaluating pneumococcal antibodies based on antibiotic sensitive targets by Nahm et al. (Nahm, M. H., D. E. Briles, and X. Yu, “Development of a multi-specificity opsonophagocytic killing assay”, Vaccine, 2000. 18(24): p. 2768-71. Multiplexed opsonophagocytic assays are widely used in the vaccine field.

Additionally two-fold multiplexed bactericidal assays for detecting the efficacy of Vibrio vaccines have been reported (Yang, J. S., et al., “A duplex vibriocidal assay to simultaneously measure bactericidal antibody titers against Vibrio cholerae O1 inaba and Ogawa serotypes”, J Microbiol Methods, 2009. 79(3): p. 289-94). An advantage of bactericidal assays over opsonophagocytosis assays which require phagocytes that kill bacteria is that bactericidal assays do not.

Multiplexed assays for detecting the efficacy of putative vaccines against Shigella are needed because this bacteria is an important cause of diarrhea worldwide, with serotypes Shigella flexneri 2a, S. flexneri 3a, and Shigella sonnei demonstrating epidemiological prevalence. Based thereon, significant development efforts are focused on Shigella lipopolysaccharide (LPS)-based vaccines.

A bactericidal assay for detecting the efficacy of Shigella vaccines has been reported (Necchi, F., A. Saul, and S. Rondini, “Development of a high-throughput method to evaluate serum bactericidal activity using bacterial ATP measurement as survival readout”, PLoS One, 2017. 12(2): p. e0172163). This bactericidal assay determines the number of killed bacteria by measuring the molecules released from the dead bacteria instead of counting the surviving bacteria. However, this assay is commercially disadvantageous because the use thereof requires specialized equipment and moreover it cannot be multiplexed.

An alternative multiplex bactericidal assay is to use a set of target bacteria strains, each expressing a unique molecule with a different fluorescent spectrum wherein the release of fluorescent molecules could be measured. However, this approach, like the above-described bactericidal assay, similarly requires specialized equipment and moreover is highly complex.

Based on the foregoing there is still a need for novel vaccines which provide protection against gram-negative bacteria, particularly those which cause human disease as well as improved, high throughput methods for evaluating the efficacy of putative vaccines designed to afford protection against different gram-negative bacterial strains to elicit “neutralizing” or “functional” antibodies against different target gram-negative bacteria.

### SUMMARY

The present disclosure relates to improved multiplex methods for evaluating the efficacy of Gram-negative vaccines and kits for use therein.

It is an object of the invention to provide a novel multiplexed serum-based bactericidal assay (SBA) for detecting the efficacy and/or specificity of a putative gram-negative bacterial vaccine to elicit neutralizing protective antibodies against 3 or more different gram-negative bacterial strains wherein the SBA detects the ability of antiserum elicited against the putative vaccine to kill 3 or more different gram-negative bacterial strains optionally of the same genus by complement-mediated cytotoxicity (CDC), and wherein the SBA is conducted in multiplex format using each of said 3 or more gram-negative bacterial strains which respectively are resistant to a different antibiotic relative to the other target gram-negative bacterial strains.

It is another object of the invention to provide a novel multiplexed SBA as above-described, which uses human or rabbit complement, e.g., baby rabbit complement (BRC).

It is another object of the invention to provide a novel multiplexed SBA as above-described, which detects the efficacy and/or specificity of a vaccine to elicit protective antibodies against 4 or more different gram-negative bacterial strains of the same genus.

It is another object of the invention to provide a novel multiplexed SBA as above-described, wherein said different gram-negative bacterial strains of the same or different genus are selected from Shigella, Salmonella, Meningococcal; E. coli, Klebsiella, Pseudomonas, Acinetobacter, Burkholderia, Clostridioides, Neisseria, Vibrio, Serratia, Campylobacter, Bordatella, Yersinia, Legionella, Haemophilus influenzae, Pasteurella multocida, Enterobacter, Bacteroides fragilis, Proteus, or Helicobacter pylori strains.

It is another object of the invention to provide a novel multiplexed SBA as above-described, wherein said different gram-negative bacterial strains comprise at least 3 or all 4 of S. flexneri 2a, S. flexneri 3a, S. sonnei, and S. flexneri 6.

It is another object of the invention to provide a novel multiplexed SBA as above-described, using a S. flexneri 2a strain resistant to streptomycin, a S. flexneri 6 strain is resistant to nalidixic acid, a S. flexneri 3a strain resistant to kanamycin, and a S. sonnei strain resistant to doxycycline, which 4 strains respectively are sensitive to the other three antibiotics (kanamycin, streptomycin, doxycycline, and nalidixic acid).

It is another object of the invention to provide a novel multiplexed SBA as above-described, wherein each of the target bacterial strains is resistant to the antibiotic used for selection but sensitive to the other antibiotic(s) used for selection of the other target strains used in the multiplexed bactericidal assay.

It is another object of the invention to provide a novel multiplexed SBA as above-described, wherein each of the antibiotic resistant strains used in the assay is resistant to an antibiotic generally not used for treatment against the target bacterial strain.

It is another object of the invention to provide a novel multiplexed SBA as above-described, wherein the growth of each target strain is not substantially affected by the resistant antibiotic at twice the working concentration used in the SBA, but growth is completely inhibited by the sensitive antibiotics at one-half the working concentrations used in the bactericidal assay.

It is another object of the invention to provide a novel multiplexed SBA as above-described, wherein the antiserum is obtained from a subject immunized with a putative multivalent vaccine developed to protect against the at least 3 different gram-negative bacterial strains.

It is another object of the invention to provide a novel multiplexed SBA as above-described, wherein the antiserum is obtained from an infant or child immunized with a putative multivalent vaccine developed to protect against different gram-negative bacterial strains e.g., Shigella, Salmonella, Meningococcal; E. coli, Klebsiella, Pseudomonas, Acinetobacter, Burkholderia, Clostridioides, Neisseria, Vibrio, Serratia, Campylobacter, Bordatella, Yersinia, Legionella, Haemophilus influenzae, Pasteurella multocida, Enterobacter, Bacteroides fragilis, Proteus, or Helicobacter pylori strains.

It is another object of the invention to provide a novel multiplexed SBA as above-described, wherein the antiserum is obtained from an elderly subject or a subject with comorbidities immunized with a putative multivalent vaccine developed to protect against the different gram-negative bacterial strains.

It is another object of the invention to provide a novel multiplexed SBA as above-described, wherein said putative vaccine contains different bacterial antigens, optionally glycoproteins, further optionally O-antigens derived from the different target bacterial strains.

It is another object of the invention to provide a novel multiplexed SBA as above-described, wherein said putative vaccine contains an antigen, optionally a glycoprotein, further optionally an O-antigen or fragment thereof which elicits protective or neutralizing antibodies against 2 or more of the target bacterial strains.

It is another object of the invention to provide a novel multiplexed SBA as above-described, wherein said putative vaccine contains a dominant epitope or consensus antigen, optionally a glycoprotein, further optionally an O-antigen or fragment thereof which elicits protective or neutralizing antibodies against 2 or more of the target bacterial strains.

It is another object of the invention to provide a novel multiplexed SBA as above-described, which includes the use of baby rabbit complement (BRC).

It is another object of the invention to provide a novel multiplexed SBA as above-described, which is used to evaluate the efficacy and/or specificity of the putative vaccine using antiserum obtained from a vaccinated subject after at least one administration of the vaccine.

It is another object of the invention to provide a novel multiplexed SBA as above-described, which is used to evaluate the efficacy and/or specificity of the putative vaccine using antiserum obtained from a vaccinated subject after multiple administrations of the vaccine, optionally wherein said multiple administrations are effected at different times.

It is another object of the invention to provide a novel multiplexed SBA as above-described, which is used to evaluate the duration of efficacy and/or specificity of a vaccine using antiserum obtained from a vaccinated subject after a period of at least 1 week, 2 weeks, 3 weeks, 1 month, 2 months, 3 months, 4 months, 5 months, 6 months, a year or longer after the last administered dosage of the vaccine.

It is another object of the invention to provide a novel multiplexed SBA as above-described, which is used to evaluate the optimal dosing interval and/or dosage of the vaccine using antiserum obtained from one or more vaccinated subjects.

It is another object of the invention to provide a kit for performing a multiplexed SBA according to any of the previous embodiments which kit comprises 3 or more frozen or lyophilized target bacteria which are respectively resistant to different antibiotics and complement, optionally rabbit complement or baby rabbit complement (BRC) and instructions for using the kit.

It is another object of the invention to provide a kit for performing a multiplexed SBA according to any of the previous embodiments, which additionally comprises other reagents such as buffers and antibiotics.

It is another object of the invention to provide a kit for performing a multiplexed SBA according to any of the previous embodiments, which additionally comprises one, two, three or all four of kanamycin, streptomycin, doxycycline, and nalidixic acid.

### BRIEF DESCRIPTION OF THE DRAWINGS

FIGS. 1A to 1D shows the antibiotic resistance of mSBA target strains. The four antibiotic resistant strains were tested for growth in multiple concentrations of the indicated antibiotics. The bars indicate detectable growth at the indicated antibiotic concentration. For each antibiotic, the black dashed vertical line represents the assay working concentration (400, 75, 2, and 2.5 mg/L for streptomycin, kanamycin, nalidixic acid, and tetracycline, respectively). The green boxes indicate 2-fold deviations from the working concentrations.

FIG. 2 shows the growth of mSBA target strains. The four target strains used in the mSBA were tested, individually, for growth on plates containing the indicated antibiotics. Each antibiotic was tested at the working assay concentration (“1×”), one-half the working assay concentration (“0.5×”), and twice the working concentration (“2×”). Growth of the strains without antibiotic is shown to the right (“No Abx”). The “1×” concentrations are 400, 75, 2, and 2.5 mg/L for streptomycin (“Strep”), kanamycin (“Kan”), nalidixic acid (“Nal Acid”), and tetracycline (“Tet”), respectively.

FIGS. 3A to 3D contains the results of experiments evaluating the robustness of mSBA Assays after different incubation time. Each graph A-D shows the killing indexes (KIs) obtained with a 120-minute incubation (X axes), a 115-minute incubation (Y axes, blue symbols), and a 125-minute incubation (Y axes, red symbols) for each indicated target strain. Each graph has a line of identity (solid black line) and lines indicating 2-fold deviations (dashed black lines). Each graph also indicates the geometric mean of the ratios (GMR) for the 20 sera for each incubation time.

FIGS. 4A to 4D contains the results of experiments evaluating the robustness of mSBA assays at different BRC concentrations. Each graph A-D shows the killing indexes (KIs) obtained with 12.5% BRC (X axes), 12% BRC (Y axes, blue symbols), and 13% BRC (Y axes, red symbols) for each indicated target strain. Each graph has a line of identity (solid black line) and lines indicating 2-fold deviations (dashed black lines). Each graph also indicates the geometric mean of the ratios (GMR) for the 20 sera for BRC concentration.

FIGS. 5A to 5D contains the results of experiments evaluating the specificity of the mSBA. Four different monoclonal antibodies (mAbs; Hflex2a4, Hflex3a5, 306/305, and Hsoni5) was tested in the mSBA, individually and in combination (mAb QC). The number of surviving bacteria (y-axis) is shown as a function of sample dilution number (x-axis). Solid and dashed black lines indicate 100% and 50% survival, respectively.

FIG. 6 contains the results of experiments evaluating the reproducibility of mSBA assays. For each mSBA target, the % CV (Y axis) is shown as a function of the geometric mean killing index (GMKI, X axis) for each serum tested. Each graph has a red dashed line indicating 50% CV and a green dashed line indicating 30% CV for reference.

FIGS. 7A to 7D contains the results of experiments evaluating the repeatability of the results of mSBA assays. For each target, the KIs for sera tested two times in the same run are shown, with the first KI shown on the X axis and the second on the Y axis. Each graph had a solid black line of identity and dashed black lines indicating 3-fold deviations from identity.

FIGS. 8A to 8B contains the results of experiments comparing KI values obtained for the 4 targets using the SFB mSBA Kit. Five samples were analyzed in the mSBA, including 4 sera and one mixture of monoclonal antibodies specific for each of the 4 target strains. Each graph has a line of identity (solid black line) and lines indicating 2-fold deviations (dashed black lines). (Top) KI values were obtained at SFB laboratory (x-axis) and in the laboratory of an external collaborator (y-axis) using the same mSBA Kit lot. (Bottom) Two different SFB mSBA kits were supplied to the external collaborator to conduct the analysis. The x-axis represents results using mSBA Kit lot A and the y-axis represents results using mSBA Kit lot B.

### DETAILED DESCRIPTION

### Definitions

Unless defined otherwise, all technical and scientific terms used herein have the same meaning as commonly understood by one of ordinary skill in the art to which this disclosure belongs.

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

It should be understood that, unless clearly indicated otherwise, in any methods disclosed or claimed herein that comprise more than one step, the order of the steps to be performed is not restricted by the order of the steps cited.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

All publications, patents, and patent applications mentioned in this specification are herein incorporated by reference to the same extent as if each individual publication, patent, or patent application was specifically and individually indicated to be incorporated by reference.

It must also be noted that, unless the context clearly dictates otherwise, the singular forms “a,” “an,” and “the” as used herein and in the appended claims include plural refence. Thus, the reference to “a bacterial cell” refers to one or more cells and equivalents thereof known to those skilled in the art, and so forth.

As used herein, the term “about” or “approximately” when used in reference to a particular recited numerical value, means that the value may vary from the recited value by no more than 10%. For example, as used herein, the expression “about 100” includes 90 and 110 and all values in between (e.g., 91, 92, 93, 99, 99.1, 99.2, 99.3, 99.4, 100, 100.8, 100.9, 101, 106, 107, 108, 109, etc.).

It is understood that aspects and embodiments of the disclosure described herein include “comprising,” “consisting,” and “consisting essentially of” aspects and embodiments. Transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It will be further understood that all transitional terms such as “comprises,” “comprising,” “including,” “having,” “containing,” “involving,” “composed of,” and the like, when used in this specification, are to be understood to be open-ended, namely, to specify the presence of stated features, integers, steps, operations, elements, and/or components, but not to preclude the presence or addition of one or more other features, integers, steps, operations, elements, components, and/or groups thereof. Only the transitional phrases “consists of” or “consisting of” shall be closed transitional phrases. The semi-closed transitional phrase “consists essentially of” or “consisting essentially of” shall be understood to specify the presence of stated features, integers, steps, operations, elements, and/or components and to allow the presence or addition of one or more other features, integers, steps, operations, elements, components, and/or groups thereof as long as they do not materially affect the basic characteristics of stated features, integers, steps, operations, elements, and/or components. For example, in some embodiments, “cells consisting essentially of T cells” may encompass a population of cells about 95% or more, about 96% or more, about 97% or more, about 98% or more, about 99% or more of which are T cells.

As used herein, the term “and/or” includes any and all combinations of one or more of the associated listed items.

As used herein the term “bacterial serotype” refers to groups within a single species of bacteria which share distinctive surface structures or antigens.

As used herein “complement-mediated cytotoxicity” or “CMC” or “complement-dependent cytotoxicity” or “CDC” generally refers to antibody-dependent complement activity associated with the killing of cells, e.g., bacterial cells. In infectious disease, both recombinant monoclonal antibodies and polyclonal antibodies generated in natural adaptive responses can mediate complement activity and elicit “protective” or “neutralizing” antibodies.

The term “killing index” or “KI” refers to the ability of a moiety, typically antibodies elicited by a putative vaccine to kill target Gram-negative bacteria.

As used herein “protective antibodies” or “neutralizing antibodies” or “protective antiserum” or “neutralizing antiserum” refers to antibodies that are responsible for defending cells from pathogens that cause disease, generally a Gram-negative bacterium. They may be produced naturally by the body as part of its immune response, and their production is triggered by both infections and vaccinations against infections. In the present invention such antibodies generally elicit killing by CMC.

As used herein the phrase “multivalent vaccine” refers to a composition that comprises one or more immunogens which confer protection and/or elicit the production of “protective antibodies” or “neutralizing antibodies” against different pathogens, generally different Gram-negative bacteria. Generally the immunogens are synthetic or naturally occurring antigens produced by target Gram-negative bacteria.

As used herein the phrase “consensus epitope” or “consensus antigen” refers to an artificial or naturally occurring antigen which elicits the production of antibodies against different pathogens, e.g., different Gram-negative bacteria.

As used herein the phrase “dominant epitope” refers to an antigen expressed by a particular pathogen, e.g., a Gram-negative bacterium, which elicits the production of “protective antibodies” or “neutralizing antibodies” against the pathogen.

The phrase “effector function” of an antibody refers to biological activities attributable to the Fc region of an antibody, which varies by antibody class or isotype. Exemplary effector functions include: complement (e.g., C1q) binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation. Generally effector function herein refers to complement-mediated cytotoxicity or killing of Gram-negative bacteria.

The term “EC50” refers to the “half maximal effective concentration”, which value measures the effectiveness of compound (e.g. an antibody) towards a biological or biochemical utility such as a pathogen, e.g., a bacterium. This quantitative measure indicates the quantity or concentration required for a particular compound to elicit a given biological process to half of the maximal response.

An “effective amount” of a composition (e.g., antigen, antiserum or vaccine composition) described herein, is at least the minimum amount required to achieve the desired therapeutic or prophylactic result, e.g., a therapeutic or prophylactic response against one or more different gram negative bacterial strains, typically of the same genus. An effective amount may vary according to inter alia disease state, age, sex, and weight of the patient, and the ability of the active ingredient (antigen, antiserum or vaccine) to elicit a desired response in the individual.

An “isolated” biological component (such as an isolated bacterial strain or antibody) refers to a component that has been substantially separated or purified away from its environment or other biological components in which the component naturally occurs.

The term “mammal” refers to any mammal, including, but not limited to, mammals of the order Rodentia, such as mice, rats, and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “OD600” is an abbreviation of two parts; ‘OD’ is short for ‘optical density’, whilst ‘600’ is in reference to the 600 nm wavelength used to measure said optical density.

The term “parenteral” or “parenterally” as used herein includes any route of administration of a compound or composition, characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

A “pharmaceutical composition” refers to a preparation in such form as to permit the biological activity of an active ingredient contained therein, such as antigens specific to one or more bacterial strains as described herein, to be effective and which preferably contains no additional components which are unacceptably toxic to a subject to which the composition would be administered.

A “pharmaceutical carrier”, as used herein, includes any and all solvents, dispersion media, coatings, antibacterial and antifungal agents, isotonic, and absorption delaying agents that are physiologically compatible. A pharmaceutically acceptable carrier includes, but is not limited to, a buffer, excipient, stabilizer, or preservative. In one embodiment, the carrier is suitable for parenteral, intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the present disclosure is contemplated. Supplementary active compounds can also be incorporated into the compositions. In some embodiments, the carrier may be a liquid, in which an active therapeutic agent is formulated. The excipient generally does not provide any pharmacological activity to the formulation, though it may provide chemical and/or biological stability, and release characteristics. Exemplary formulations can be found, for example, in Remington's Pharmaceutical Sciences, Gennaro, A. editor, 19th edition, Philadelphia, PA: Williams and Wilkins (1995), which is incorporated by reference.

The term “subject” as used herein may be any living organisms, preferably a mammal, more preferably a human. In some embodiments, the subject is a primate such as a human. In some embodiments, the primate is a monkey or an ape. The subject can be male or female and can be any suitable age, including infant, juvenile, adolescent, adult, and geriatric subjects. The subject may also be referred to as “patient” in the art. The subject may have a disease or may be healthy.

As used herein, the term “prevent,” “prevention,” or “prophylaxis” generally refers to a clinical procedure, e.g., vaccination, which prevents or inhibits a subject from developing a disease or a condition, or which prevents one or more conditions or symptoms of a disease. Herein the disease or condition typically refers to one caused by infection with a Gram negative bacterium, e.g., Shigella, Salmonella, Meningococcal; E. coli, Klebsiella, Pseudomonas, Acinetobacter, Burkholderia, Clostridioides, Neisseria, Vibrio, Serratia, Campylobacter, Bordatella, Yersinia, Legionella, Haemophilus influenzae, Pasteurella multocida, Enterobacter, Bacteroides fragilis, Proteus, or Helicobacter pylori strains.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. Herein the disease or condition typically refers to one caused by infection with a Gram negative bacterium, e.g., Shigella, Salmonella, Meningococcal; E. coli, Klebsiella, Pseudomonas, Acinetobacter, Burkholderia, Clostridioides, Neisseria, Vibrio, Serratia, Campylobacter, Bordatella, Yersinia, Legionella, Haemophilus influenzae, Pasteurella multocida, Enterobacter, Bacteroides fragilis, Proteus, or Helicobacter pylori strains.

The term “working stock” or “WS” refers to stocks comprising target bacterial strains.

### DESCRIPTION

Shigellosis is a gastrointestinal disease caused by the Gram-negative bacteria Shigella which is characterized by watery or bloody diarrhea (Lampel, K. A., S. B. Formal, and A. T. Maurelli, “A Brief History of Shigella”, EcoSal Plus, 2018. 8(1)). Shigella includes four species—S. dysenteriae, S. flexneri, S. boydii, and S. sonnei, and each species except S. sonnei have multiple strains, each expressing a unique LPS serotype (Levine, M. M., et al., “Clinical trials of Shigella vaccines: two steps forward and one step back on a long, hard road”, Nat Rev Microbiol, 2007. 5(7): p. 540-53; Perepelov, A. V., et al., “Shigella flexneri O-antigens revisited: final elucidation of the O-acetylation profiles and a survey of the O-antigen structure diversity”, FEMS Immunol Med Microbiol, 2012. 66(2): p. 201-10.

Shigellosis represents a large disease burden as it results in ^{˜}212,000 annual deaths, including ^{˜}63,000 in children under 5 years of age, primarily in low- and middle-income countries (LMIC) (Khalil, I. A., et al., “Morbidity and mortality due to shigella and enterotoxigenic Escherichia coli diarrhoea: the Global Burden of Disease Study 1990-2016”, Lancet Infect Dis, 2018. 18(11): p. 1229-1240; Giersing, B. K., et al., “Clinical and regulatory development strategies for Shigella vaccines intended for children younger than 5 years in low-income and middle-income countries”, Lancet Glob Health, 2023. 11(11): p. e1819-e1826).

Shigellosis damages the intestinal epithelium and disrupts the epithelial barrier (Lampel, K. A., S. B. Formal, and A. T. Maurelli, “A Brief History of Shigella”, EcoSal Plus, 2018. 8(1); Schnupf, P. and P. J. Sansonetti, “Shigella Pathogenesis: New Insights through Advanced Methodologies”. Microbiol Spectr, 2019. 7(2)) and may produce long term consequences such as growth stunting in young children (Giersing, B. K., et al., “Clinical and regulatory development strategies for Shigella vaccines intended for children younger than 5 years in low-income and middle-income countries”, Lancet Glob Health, 2023. 11(11): p. e1819-e1826); Bagamian, K. H., et al., “Shigella and childhood stunting: Evidence, gaps, and future research directions”, PLoS Negl Trop Dis, 2023. 17(9): p. e0011475; and Libby, T. E., et al., “Consequences of Shigella infection in young children: a systematic review”, Int J Infect Dis, 2023. 129: p. 78-95). Since Shigella infection can occur after ingestion of a low infectious dose (^{˜}100 bacteria) (Lampel, K. A., S. B. Formal, and A. T. Maurelli, “A Brief History of Shigella”, EcoSal Plus, 2018. 8(1)), civilians and military personnel may easily contract shigellosis while traveling in countries where Shigella is endemic.

In addition, the prevalence of antibiotic resistant Shigella is increasing and, even after a successful treatment with antibiotics, shigellosis can cause cognitive impairment (Nasrin, D., et al., “Pathogens Associated With Linear Growth Faltering in Children With Diarrhea and Impact of Antibiotic Treatment: The Global Enteric Multicenter Study”, J Infect Dis, 2021. 224(12 Suppl 2): p. S848-S855; MacLennan, C. A. and A. D. Steele, “Frontiers in Shigella Vaccine Development”, Vaccines (Basel), 2022. 10(9)); or reactive arthritis (Hannu, T., et al., “Reactive arthritis attributable to Shigella infection: a clinical and epidemiological nationwide study”, Ann Rheum Dis, 2005. 64(4): p. 594-8). Consequently, there is a great need for developing effective vaccines against shigellosis (MacLennan, C. A., et al., “Critical Needs in Advancing Shigella Vaccines for Global Health”, J Infect Dis, 2022. 225(9): p. 1500-1503).

Since the discovery of Shigella in 1896 (Lampel, K. A., S. B. Formal, and A. T. Maurelli, “A Brief History of Shigella”, EcoSal Plus, 2018. 8(1)), multiple approaches have been tested to develop vaccines, including whole cell killed and live attenuated bacteria, bacterial outer membrane vesicle preparations and O-antigen glycoconjugates produced by bioconjugation or chemical conjugation methods (MacLennan, C. A., et al., “Critical Needs in Advancing Shigella Vaccines for Global Health”, J Infect Dis, 2022. 225(9): p. 1500-1503). The O-antigen, which is the structurally variable distal part of LPS, determines the serotype of Shigella (Levine, M. M., et al., “Clinical trials of Shigella vaccines: two steps forward and one step back on a long, hard road”, Nat Rev Microbiol, 2007. 5(7): p. 540-53; Perepelov, A. V., et al., “Shigella flexneri O-antigens revisited: final elucidation of the O-acetylation profiles and a survey of the O-antigen structure diversity”, FEMS Immunol Med Microbiol, 2012, 66(2): p. 201-10).

The antibodies to O-antigens induced with a glyco-conjugate have shown protection against severe shigellosis in a controlled human infection model (CHIM) using S. flexneri serotype 2a (Talaat, K. R., et al., “Human challenge study with a Shigella bioconjugate vaccine: Analyses of clinical efficacy and correlate of protection”, EBioMedicine, 2021. 66: p. 103310). However, the O-antigen antibodies provide serotype-specific protection and therefore, protection against the majority of circulating Shigella strains requires a glycoconjugate vaccine with O-antigens from multiple serotypes.

According to the Global Enteric Multicenter Study (GEMS), a quadrivalent vaccine containing three S. flexneri serotypes (2a, 3a, and 6) and S. sonnei would protect against ^{˜}75% of global strains and provide up to 93% coverage through cross-reactive epitopes (Livio, S., et al., “Shigella isolates from the global enteric multicenter study inform vaccine development”, Clin Infect Dis, 2014, 59(7): p. 933-41).

Antibodies targeting the O-antigen can kill targeted Shigella by complement activation. However, some antibodies to O-antigen appears to be non-functional: they bind the target antigen without killing (Lin, J., et al., “Monoclonal Antibodies to Shigella Lipopolysaccharide Are Useful for Vaccine Production”, Clin Vaccine Immunol, 2016. 23(8): p. 681-8).

Consequently, serum bactericidal assays (SBA) have been developed to quantitate the functional O-antigen antibodies (Necchi, F., A. Saul, and S. Rondini, “Development of a high-throughput method to evaluate serum bactericidal activity using bacterial ATP measurement as survival readout”, PLoS One, 2017. 12(2): p. e0172163; Nahm, M. H., et al., “Development, Interlaboratory Evaluations, and Application of a Simple, High-Throughput Shigella Serum Bactericidal Assay”, mSphere, 2018. 3(3) and SBA results correlate with protection against shigellosis in CHIM 2a (Talaat, K. R., et al., “Human challenge study with a Shigella bioconjugate vaccine: Analyses of clinical efficacy and correlate of protection”, EBioMedicine, 2021. 66: p. 103310).

A simple, robust, reproducible, and high-throughput SBA should expedite the conjugate vaccine development by facilitating the evaluation of immune responses to vaccine candidates. To facilitate the development of multivalent, i.e., quadrivalent Shigella vaccines (MacLennan, C. A., et al., The Shigella Vaccines Pipeline. Vaccines (Basel), 2022. 10(9); Rossi, O., et al., “A next-generation GMMA-based vaccine candidate to fight shigellosis”, NPJ Vaccines, 2023. 8(1): p. 130); the present inventors have developed a multivalent SBA (mSBA) targeting the four strains: S. flexneri 2a, 3a, 6, and S. sonnei, and have characterized the mSBA following the ICH recommendation (ICH, ICH Topic Q2B Validation of Analytical Methods, in ICH Harmonised Tripartite Guideline: Validation of Analytical Methods, I.-T. Coordination, Editor. 1997, ICH: London. p. 1-9).

Examples are provided below to illustrate exemplary embodiments of the present disclosure. However, these examples are not meant to constrain the present invention to any particular application or theory of operation.

### EXAMPLES

### Materials and Methods

The following materials and methods were used in the examples.

### Monoclonal Antibodies and Human Serum

Four serotype-specific mouse monoclonal antibodies (mAb) were used to assess mSBA specificity: Hflex2a4, Hflex3a5, and Hsoni5 specific for S. flexneri 2a, S. flexneri 3, and S. sonnei respectively, were obtained from SunFire Biotechnologies LLC (Birmingham, AL); and mAb 306/305, specific for S. flexneri 6, was obtained from Bio-Rad Laboratories (Hercules, CA).

Deidentified human sera were obtained from Dr. Marcela Pasetti's repository at the University of Maryland, from Dr. K. H. Kim's repository at Ewha Women's University (Seoul, Korea), and commercially from BioIVT (Westbury, NY) and Innovative Research (Novi, MI).

### Target Strains

Antibiotic resistant target strains needed were derived from S. sonnei strain Moseley and S. flexneri strains 2457T (serotype 2a), J17B (serotype 3a) and CCH060 (serotype 6). Strains 2457T, J17B and Moseley strains are public strains and were obtained from Dr. Moon Nahm at the University of Alabama at Birmingham (Birmingham, AL), and CCH060 was obtained from Dr. Kaminski at Walter Reed Army Institute of Research in Silver Spring MD.

Antibiotic resistance screening found that Moseley is naturally resistant to doxycycline but sensitive to other three antibiotics (kanamycin, streptomycin and nalidixic acid). Strains 2457T (S. flexneri 2a) and SNP0006 (S. flexneri 6) were made resistant to streptomycin and nalidixic acid, respectively, by natural selection as previously described (Burton, R. L. and M. H. Nahm, “Development and validation of a fourfold multiplexed opsonization assay (MOPA4) for pneumococcal antibodies”, Clin Vaccine Immunol, 2006. 13(9): p. 1004-9). Strain J17B (S. flexneri 3a) was made kanamycin resistant by transduction with a nadC:kan fusion gene with P1 phage (Prunier, A. L., et al., “Genetic structure of the nadA and nadB antivirulence loci in Shigella spp.”, J Bacteriol, 2007. 189(17): p. 6482-6), which was obtained from a S. flexneri 2a strain (BS655) (personal communication with Dr. C. Turnbough at UAB). After selection, all targets were made resistant to the antibiotic used for selection but sensitive to the other three antibiotics, and all strains expressed the indicated LPS serotypes as determined with the serotype-specific monoclonal antibodies by flow cytometry (data not shown).

To prepare working stocks for the target strains, primary stocks were streaked onto blood agar plates and incubated at 37° C./5% CO₂. After overnight culture, multiple colonies were transferred to liquid culture medium (Todd-Hewitt broth with 0.5% yeast extract) and grown at 37° C. until cultures reached an OD600 of 0.5-1.4. After reaching the desired OD, glycerol was added to a final concentration of 16%, and 0.5-ml aliquots were prepared and stored at ≤−60° C. until needed. Each WS was tested to confirm the antibiotic sensitivity/resistance profile and to determine the optimal dilution of bacteria to use for the assay.

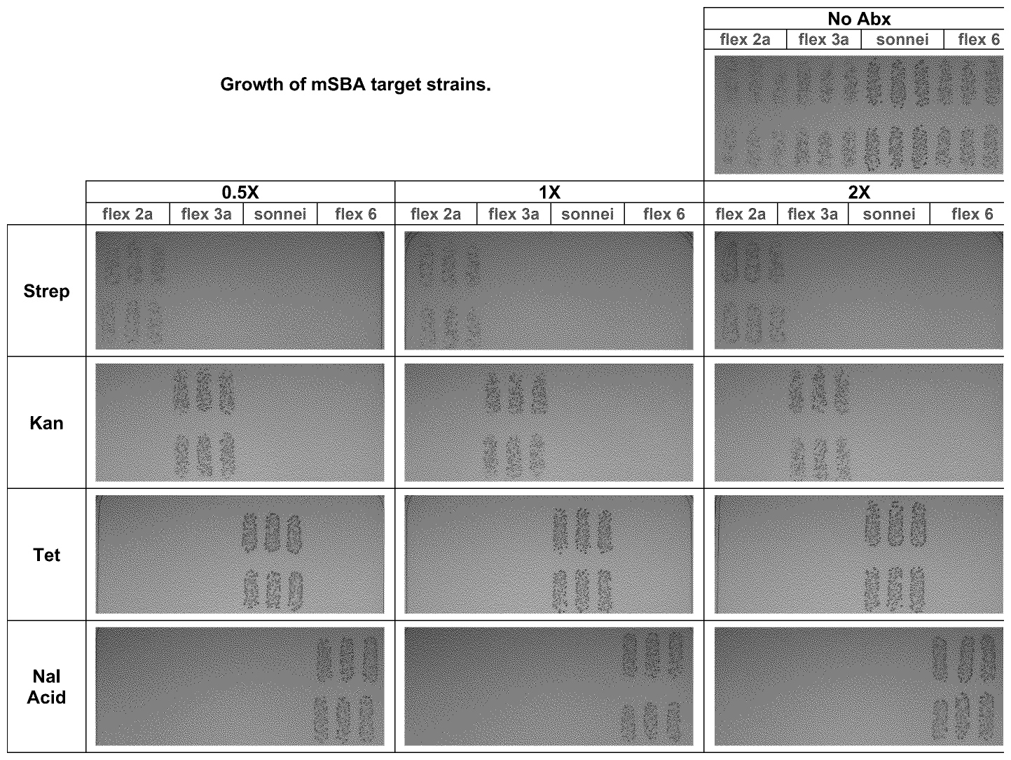

All target strains were tested individually in assay conditions for growth on plates containing antibiotics. As shown in FIG. 2, only the expected strain survives in the presence of each antibiotic. It is important to note that the sensitive strains do not survive even at one-half assay concentrations, and the resistant strains survive well even at twice the assay concentrations. This 2-fold window provides robustness to the assay utilizing these strains.

mSBA Procedure

Prepare SAB (Shigella Assay Buffer) by mixing 80 ml of sterile water, 10 ml of 10×HBSS with Ca++ and Mg++, and 10 ml of 1% gelatin. Obtain a microtiter plate with U bottom. The plate has 96 wells in 8 rows (labeled A through H) and 12 columns (labeled 1 through 12). Add 20 ul of SAB to each well in rows A through G. Add 30 ul of serum samples to be analyzed in row H. Assays are done in duplicates. So one sample is added to the wells in row H of columns 1 and 2. Another sample is added to columns 3 and 4 of row H etc. If the sample is projected to have a high titer, samples may be pre-diluted (1-100 fold) in SAB. Perform 3 fold serial dilutions from row H to row A as following. Transfer 10 ul of sample in row H to G. Mix the samples in row G well. Then transfer 10 ul from row G to row F etc. After transferring to wells in row A, pick up the 10 ul and discard 10 ul. After this step, all wells contain 20 ul.

In these methods an aliquot of each of the 4 targets is washed and resuspended in Shigella Assay Buffer (SAB, Hanks' Balanced Salt Solution [with Ca++/Mg++] and 0.2% gelatin). A mixture of the 4 target strains in SAB was prepared by diluting each target strain using a pre-determined dilution factor, designed to yield ^{˜}70-150 colony forming units (CFU) per spot at the completion of the assay. To each well of the assay plate, 10 microliters of the target bacteria mixture was added, followed by addition of 50 microliters of 20% baby rabbit complement (BRC) obtained from Pel-Freez Biologicals (Rogers, AR).

After incubation at 37° C. in room air for 120 minutes followed by incubation on ice for 20 minutes, 10 μl aliquots of reaction mixture from each well were spotted onto four Todd-Hewitt yeast agar plates (THYA, Todd Hewitt broth with 0.5% yeast extract and 1.5% agar) containing 100 mg/L 2,3,5-triphenyltetrazolium chloride (TTC) and a selective antibiotic. The selective antibiotics were doxycycline, streptomycin, kanamycin, and nalidixic acid. After overnight incubation of THYA plates at 29° C. to 32° C. (optimal overnight temperature was target strain dependent), the surviving colonies (CFU) in each spot were enumerated using NIST's Integrated Colony Enumerator (NICE) software (Clarke, M. L., et al., Low-cost, high-throughput, automated counting of bacterial colonies. Cytometry A, 2010. 77(8): p. 790-7). The surviving CFUs were converted to an SBA killing index (KI) using linear interpolation of 50% killing (Wang, D., et al., “A four-parameter logistic model for estimating titers of functional multiplexed pneumococcal opsonophagocytic killing assay”, J Biopharm Stat, 2008. 18(2): p. 307-25).

### Example 1: Design of mSBA Against Target Bacterial Strains

The target bacteria were resistant to one antibiotic and sensitive to the other three antibiotics, as shown in Table 1. The working concentrations of the 4 antibiotics were chosen such that the growth of each target strain was not affected by the resistant antibiotic at twice the working concentration, but growth was completely inhibited by the sensitive antibiotics at one-half the working concentrations.

TABLE 1
mSBA Target Strains. For each strain, the antibiotic sensitivity
is indicated (S = Sensitive, R = Resistant).

| Strain | Parent | | | Antibiotic Sensitivity | | | |
|---|---|---|---|---|---|---|---|
| Name | Strain | Species | Serotype | Dox | Strep | Kan | NA |
| Moseley | N/A | sonnei | N/A | R | S | S | S |
| SNP0001 | 2457T | flexneri | 2a | S | R | S | S |
| SNP0004 | J17B | flexneri | 3a | S | S | R | S |
| SNP0006 | CCH060 | flexneri | 6 | S | S | S | R |

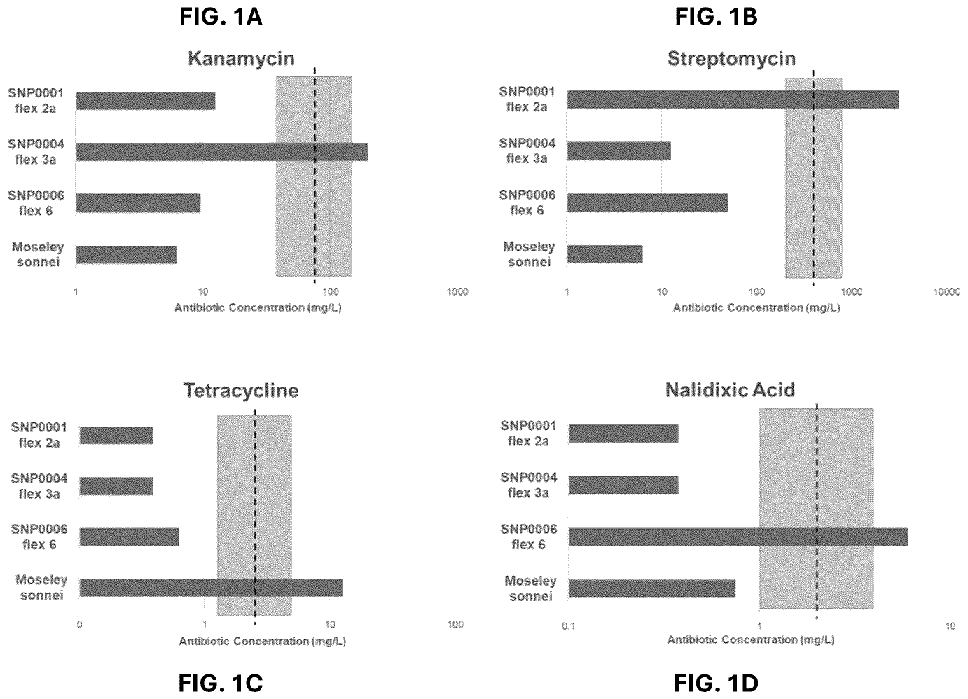

As shown in FIGS. 1A-1D, each target strain was tested for resistance to kanamycin, streptomycin, tetracycline, and nalidixic acid. Based on those results, the operational assay concentrations were initially set at 75, 400, 2.5, and 2 mg/L (kanamycin, streptomycin, tetracycline, and nalidixic acid, respectively). These concentrations (indicated by dashed black lines in FIGS. 1A-1D) provided at least a 2-fold error margin in antibiotic concentration as the resistant strain was resistant at two times the assay concentration (right edges of green boxes in FIGS. 1A-1D) and the sensitive strains were sensitive at one half the assay concentration (left edges of green boxes in FIGS. 1A-1D).

As shown in FIG. 2, all target strains were tested individually in assay conditions for growth on plates containing antibiotics. As further shown in FIG. 2, only the expected strain survives in the presence of each antibiotic. It is important to note that the sensitive strains do not survive even at one-half assay concentrations, and the resistant strains survive well even at twice the assay concentrations. This 2-fold window provides robustness to the assay utilizing these strains.

### Example 2: mSBA Robustness

To be used for clinical trials, an analytical assay must demonstrate adequate robustness. An assay's robustness herein refers to its capacity to remain unaffected by small, but deliberate, variations in method parameters and provides an indication of its reliability during normal usage” (European Medicines Agency. ICH Topic Q 2 (R1) Validation of Analytical Procedures: Text and Methodology, Step 5, Note for Guidance on Validation of Analytical Procedures: Text and Methodology. 1995). The robustness of the mSBA was assessed by determining the effect of different incubation times and baby rabbit complement (BRC) concentrations on assay performance.

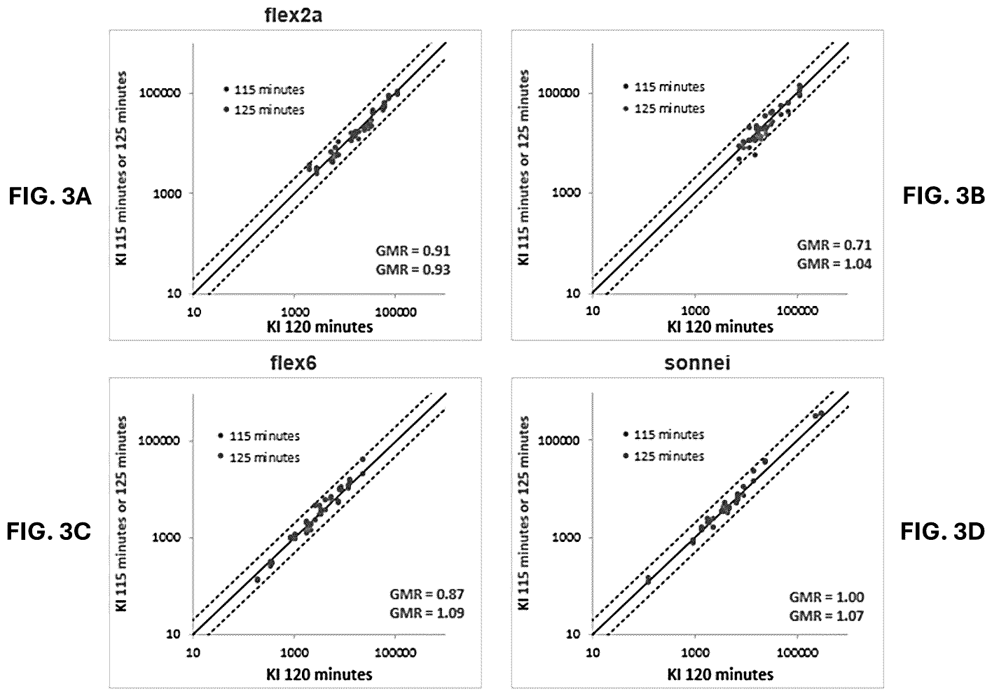

The mSBA procedure includes a 120-minute incubation step for complement activation and subsequent bacterial lysis to occur. To determine the effect of assay incubation time, 10 sera were tested using 115-, 120-, and 125-minute incubation times in side-by-side experiments. As shown in FIGS. 3A-D, all results obtained with the two alternate incubation times were within 2-fold of those obtained using the standard 120-minute incubation for flex2a, flex6, and sonnei. The results were consistent across the range of observed KIs. Additionally, the geometric mean of the ratios (GMR) was close to 1.0 (range 0.87 to 1.09) for those targets.

For flex3a, all results obtained with the 125-minute incubation were within 2-fold of those obtained using the 120-minute incubation, and the GMR was 1.04. The results obtained with the 115-minute incubation were generally lower than those using the 120-minute incubation, with one result deviating by more than 2-fold. The trend was noted across the range of observed KIs. The GMR for the 115-minute incubation was 0.71, indicating that the 115-minute incubation yielded KIs that were about 30% lower, on average.

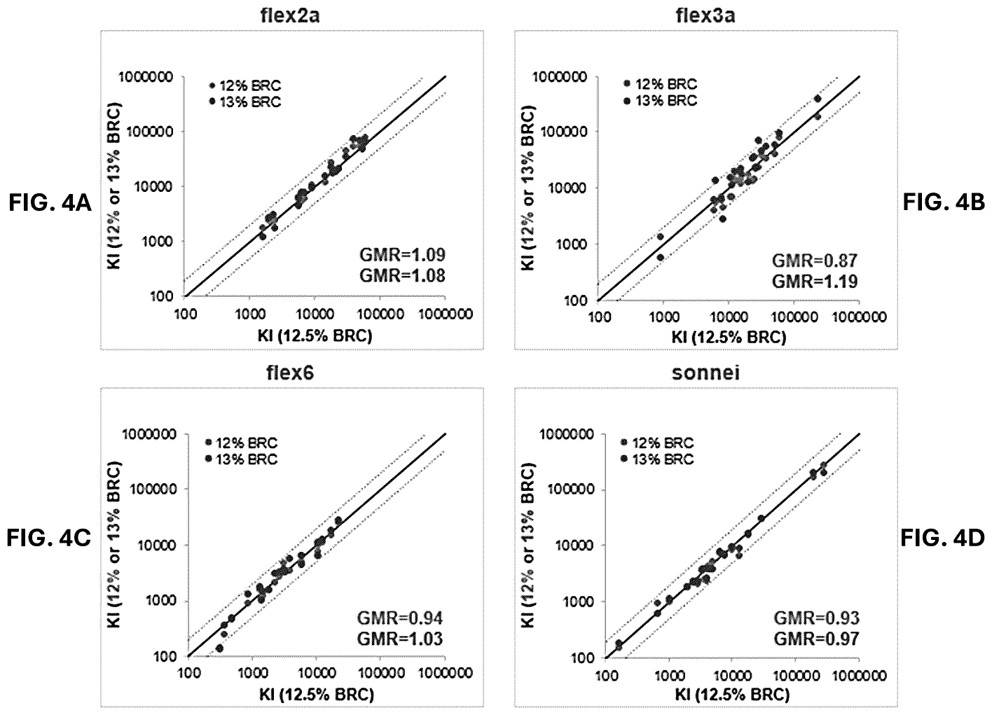

The mSBA uses a final baby rabbit complement (BRC) concentration of 12.5%. To further assess the robustness of the mSBA, the impact of different BRC concentrations was evaluated. BRC concentrations of 12%, 12.5%, and 13% were tested in side-by-side experiments. As shown in FIGS. 4A-D, the results obtained using 12% and 13% BRC were comparable to those obtained using 12.5% BRC. For flex2a, flex6, and sonnei, 2 results deviated by more than 2-fold. Both results were for flex6, and both alternate BRC concentrations yielded lower results than the 12.5% BRC. The GMRs for these targets were quite close to 1.0 (range, 0.93 to 1.09).

For flex3a, all results using 12% BRC were within 2-fold of those obtained with 12.5% BRC. With 13%, there were 3 results that differed by more than 2-fold from those obtained using 12.5% BRC, with 2 of the results higher and 1 result lower. For flex3a, the GMRs for both alternate BRC concentrations were close to 1.0 (0.87 and 1.19).

### Example 3: mSBA Specificity

Specificity in the context of the present invention can be defined as the ability of a moiety, e.g., a monoclonal antibody or antiserum to specifically kill a particular target bacterium strain. To assess the specificity of the mSBA, the assay was performed with the four monoclonal antibodies (mAb): Hflex2a4, Hflex3a5, 306/305, and Hsoni5 (specific for S. flexneri 2a, S. flexneri 3a, S. flexneri 6, and S. sonnei, respectively).

In these experiments the four different monoclonal antibodies (mAbs; Hflex2a4, Hflex3a5, 306/305, and Hsoni5) were each tested in the mSBA, individually and in combination (mAb QC). The number of surviving bacteria (y-axis) is shown as a function of sample dilution number (x-axis). Solid and dashed black lines indicate 100% and 50% survival, respectively.

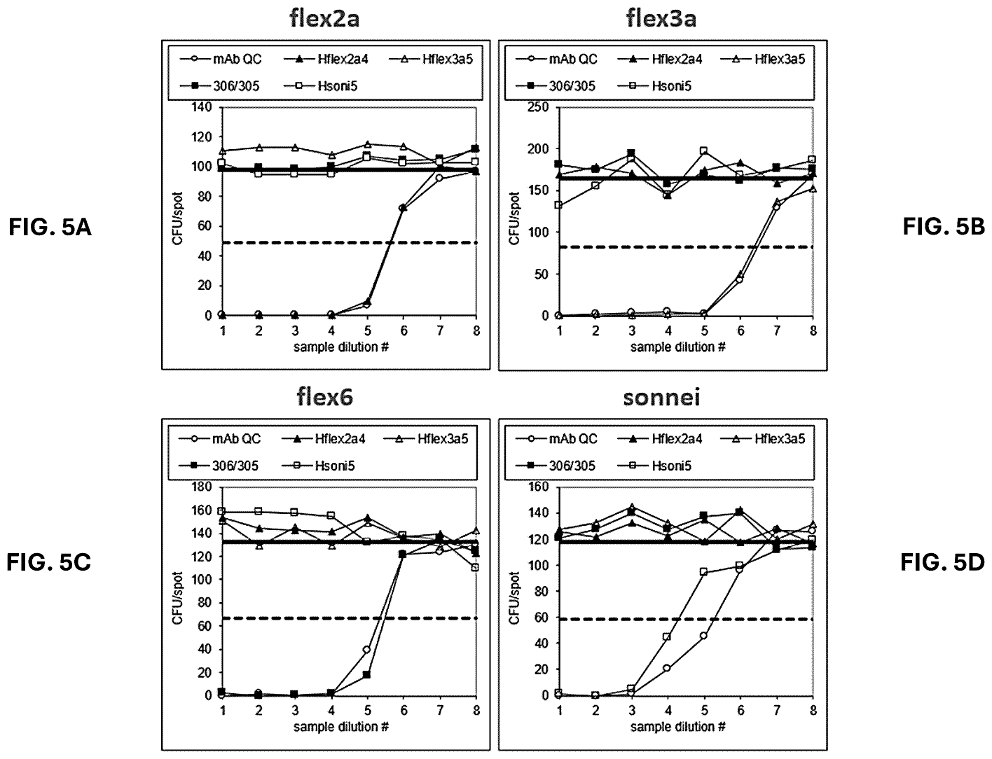

The results in FIG. 5A-D show that each mAb specifically killed the target strain expressing the targeted LPS with little or no killing of the other target strains, indicating the mSBA is serotype specific.

### Example 4: mSBA Precision (Reproducibility)

Reproducibility was determined by testing a panel of 30 human sera four times over a ^{˜}1 month period. After removing results outside of the technical limits, the geometric mean killing index (GMKI) and coefficient of variation (CV) were calculated for each sample. CV was calculated using the formula below.

$$\% CV = \sqrt{{e}^{S D {T}^{2}} - 1} \times 100$$

where SDT is the standard deviation of the logₑ-transformed KIs.

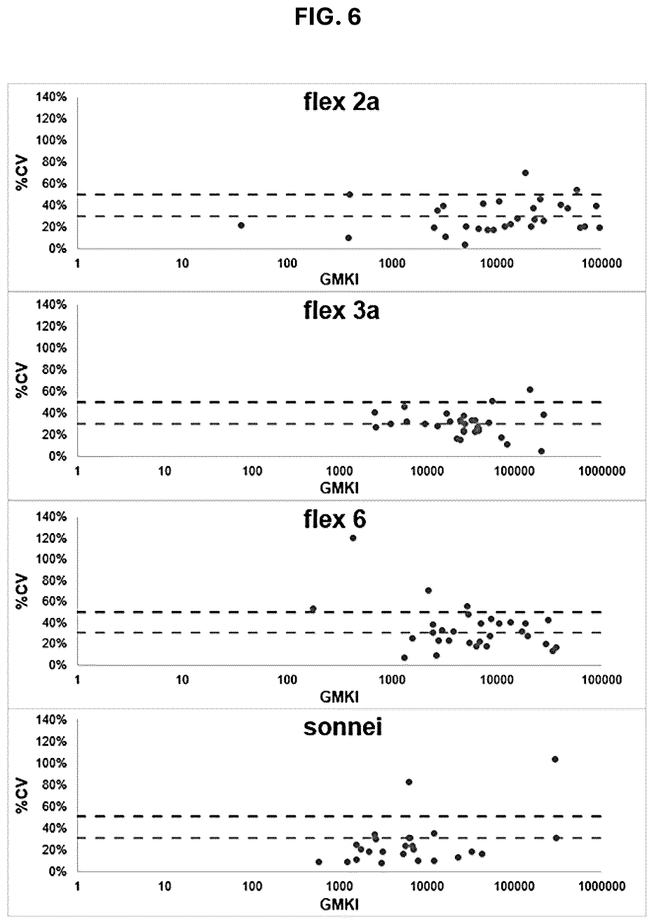

Reproducibility results are shown in Table 2 below, and in FIG. 6. In FIG. 6 for each mSBA target, the % CV (Y axis) is shown as a function of the geometric mean killing index (GMKI, X axis) for each serum tested. Each graph has a red dashed line indicating 50% CV and a green dashed line indicating 30% CV for reference.

Table 2. mSBA Reproducibility. Results from reproducibility assessments are shown below. The killing indexes (KIs) from the four individual runs, the geometric mean killing index (GMKI) of the four runs, and coefficient of variation (CV) are included. KIs below the lower technical limit are indicated as “<TL” and KIs above the upper technical limit are indicated as “>TL”. NR, no result.

| | flex 2a | | | | | | flex 3a | | | | | |
|---|---|---|---|---|---|---|---|---|---|---|---|---|
| | Run 1 | Run 2 | Run 3 | Run 4 | GMKI | CV | Run 1 | Run 2 | Run 3 | Run 4 | GMKI | CV |
| S01 | 8778 | 9924 | 7855 | 11484 | 9415 | 16% | >TL | >TL | >TL | >TL | NA | NA |
| S02 | 356 | 371 | 415 | 439 | 394 | 10% | 3281 | 3551 | 3383 | 6027 | 3926 | 29% |
| S03 | 5378 | 4124 | 4826 | 6662 | 5168 | 20% | 24869 | 18057 | 24730 | 22591 | 22380 | 15% |
| S04 | 15700 | 9998 | 6168 | 13335 | 10660 | 43% | 3117 | 1915 | 2585 | 3426 | 2697 | 26% |
| S05 | 52600 | 40605 | 24375 | 56420 | 41399 | 39% | >TL | 230336 | 104620 | >TL | 155235 | 60% |
| S06 | 14843 | 12179 | 11256 | 18381 | 13907 | 22% | 41753 | 32371 | 29316 | 46686 | 36880 | 22% |
| S07 | 38 | 28 | 46 | 37 | 37 | 21% | 2374 | 3411 | 1557 | 3543 | 2585 | 40% |
| S08 | 2966 | 3436 | 3167 | 3807 | 3330 | 11% | 46055 | 74390 | 37282 | 56219 | 51766 | 30% |
| S09 | 6663 | 6716 | 5498 | 8489 | 6760 | 18% | 5961 | 6370 | 3981 | 8221 | 5937 | 31% |
| S10 | 204 | 488 | 439 | 586 | 400 | 49% | >TL | 21709 | 38267 | 26733 | 28109 | 29% |
| S11 | 22554 | 16437 | 22740 | 26634 | 21768 | 20% | 17225 | 12779 | 25572 | 22717 | 18910 | 32% |
| S12 | 2249 | 3301 | 1981 | 4075 | 2782 | 34% | NR | 27508 | 21678 | 33654 | 27175 | 22% |
| S13 | >TL | 109772 | 84458 | NR | 96287 | 19% | 29960 | 29563 | 17121 | NR | 12690 | 33% |
| S14 | >TL | 73949 | 51355 | 67165 | 63419 | 19% | 33332 | 54644 | 37664 | 25700 | 36439 | 32% |
| S15 | 12037 | 12556 | 9479 | 15459 | 12199 | 20% | >TL | 214003 | 202189 | NR | 208012 | 4% |
| S16 | 47830 | 47693 | 31337 | 75368 | 48178 | 37% | 12762 | 19638 | NR | 26922 | 16950 | 39% |
| S17 | 80362 | 87000 | 63669 | 152935 | 90835 | 38% | 21045 | 21107 | 25760 | 44394 | 26697 | 36% |
| S18 | 2692 | 2103 | 2393 | 3243 | 2575 | 19% | 21375 | 25134 | 22579 | 29534 | 24465 | 14% |
| S19 | 74879 | 75164 | 51973 | 80821 | 69730 | 20% | 37458 | 52053 | 28883 | 43828 | 39637 | 25% |
| S20 | 7206 | 9855 | 7146 | 9286 | 8285 | 17% | 48714 | 42631 | 27009 | 39064 | 38474 | 26% |
| S21 | 19768 | 24472 | 15671 | 35878 | 22837 | 36% | 21400 | 29016 | 24049 | 35733 | 27028 | 23% |
| S22 | 18051 | 31228 | 19633 | 26574 | 23288 | 26% | 86605 | NR | 74721 | 91298 | 83910 | 10% |
| S23 | 5217 | 5146 | 5216 | 4843 | 5103 | 4% | 42574 | 46869 | 28776 | 46221 | 40362 | 23% |
| S24 | 25057 | 28736 | 23363 | 7448 | 18814 | 69% | 38265 | 37169 | 20716 | 40877 | 33128 | 32% |
| S25 | 41732 | 15128 | 24732 | 31318 | 26444 | 45% | >TL | 71237 | 63079 | 86937 | 73103 | 16% |
| S26 | 2231 | 4604 | 2284 | 4009 | 3114 | 39% | 6543 | 10412 | 9674 | 12972 | 9616 | 29% |
| S27 | 55328 | 49252 | 37236 | 121150 | 59213 | 54% | >TL | 212689 | 156267 | 320380 | 220002 | 37% |
| S28 | 23990 | NR | 25540 | 38228 | 28612 | 26% | 10096 | 17563 | 11100 | 15812 | 13282 | 27% |
| S29 | 15075 | 13224 | 13828 | 23895 | 16020 | 28% | 3618 | 7980 | 4273 | 8431 | 5679 | 45% |
| S30 | 4919 | 7348 | 6963 | 12853 | 7542 | 41% | 43265 | NR | 42187 | 97692 | 56285 | 51% |

| | flex 6 | | | | | | sonnei | | | | | |
|---|---|---|---|---|---|---|---|---|---|---|---|---|
| | Run 1 | Run 2 | Run 3 | Run 4 | GMKI | CV | Run 1 | Run 2 | Run 3 | Run 4 | GMKI | CV |
| S01 | 112 | 475 | 895 | 748 | 435 | 119% | <TL | <TL | <TL | <TL | <TL | NA |
| S02 | 1863 | 2012 | 3452 | 2898 | 2475 | 30% | <TL | <TL | <TL | <TL | <TL | NA |
| S03 | 22483 | 9047 | 11833 | 14991 | 13782 | 40% | 7480 | 4417 | 5466 | 6130 | 5768 | 22% |
| S04 | 7492 | 5294 | 5031 | 4887 | 5588 | 20% | <TL | <TL | <TL | <TL | <TL | NA |
| S05 | 29120 | 33843 | 34544 | 22985 | 29742 | 19% | 3136 | 3396 | 2916 | 2991 | 3104 | 7% |
| S06 | 21191 | 18625 | 21416 | 11261 | 17565 | 31% | 2224 | 3362 | 1972 | 3409 | 2663 | 29% |
| S07 | 110 | 128 | 270 | 283 | 181 | 52% | <TL | <TL | <TL | <TL | <TL | NA |
| S08 | 5115 | 6769 | 7443 | 6973 | 6511 | 17% | 31613 | 43427 | 30491 | 30580 | 33636 | 17% |
| S09 | NR | NR | 2576 | 2881 | 2724 | 8% | 557 | 644 | 545 | 594 | 584 | 8% |
| S10 | 3088 | 3034 | 4843 | 3421 | 3530 | 22% | <TL | <TL | <TL | <TL | <TL | NA |
| S11 | 5323 | 5317 | 8483 | 11265 | 7212 | 38% | >TL | 540964 | NR | NR | 297486 | 102% |
| S12 | 3023 | 2493 | 3780 | 2301 | 2846 | 22% | 50755 | 44708 | 35576 | 45429 | 43761 | 15% |
| S13 | 1349 | 1393 | 1244 | NR | 1267 | 6% | 12418 | 13415 | 11160 | NR | 7099 | 9% |
| S14 | 3392 | 3133 | 5485 | NR | 3877 | 31% | 6126 | 8958 | 6059 | NR | 6928 | 23% |
| S15 | 1584 | 1245 | 2218 | 1483 | 1596 | 25% | 6475 | 5872 | 4624 | 9457 | 6386 | 30% |
| S16 | 6831 | 7409 | 12145 | 9217 | 8676 | 26% | >TL | 380316 | NR | >TL | 309341 | 30% |
| S17 | 1577 | 1581 | 4720 | NR | 2275 | 70% | 8262 | 11375 | 17880 | 14491 | 12492 | 34% |
| S18 | 2137 | 1940 | 4282 | 2215 | 2504 | 37% | 22157 | 23193 | 20579 | 27264 | 23172 | 12% |
| S19 | 4666 | 7291 | 8458 | 2681 | 5270 | 55% | 6093 | 9449 | 6747 | 7090 | 7244 | 19% |
| S20 | 30390 | 40246 | 43233 | 35667 | 37058 | 16% | 5144 | 9898 | 5941 | 5794 | 6470 | 30% |
| S21 | 5217 | 7846 | 8441 | 7187 | 7059 | 21% | 1674 | 1906 | 1139 | 1861 | 1613 | 24% |
| S22 | 14311 | 25311 | 24000 | 18266 | 19962 | 27% | 5634 | 5758 | 4415 | 6221 | 5464 | 15% |
| S23 | 40568 | 37338 | 31522 | 31187 | 34933 | 13% | 2498 | 2528 | 1736 | 2253 | 2229 | 18% |
| S24 | 5279 | 3156 | 9382 | 5808 | 5489 | 47% | 9075 | 7411 | 7901 | 7879 | 8044 | 9% |
| S25 | 6591 | 7709 | 9214 | 9594 | 8187 | 17% | 1903 | 3336 | 2013 | 3492 | 2585 | 33% |
| S26 | 2020 | 3014 | 3202 | 4306 | 3027 | 32% | 1167 | 1331 | 1179 | 1374 | 1259 | 8% |
| S27 | 10774 | 6322 | 14699 | 13019 | 10685 | 39% | 2565 | 3363 | 3114 | 3848 | 3189 | 17% |
| S28 | 5541 | 11029 | NR | 11540 | 8901 | 43% | 1460 | 1928 | 1646 | 2264 | 1800 | 19% |
| S29 | 12253 | 15470 | 25181 | 26365 | 18835 | 39% | 1636 | 1412 | 1524 | 1780 | 1582 | 10% |
| S30 | 20893 | NR | 31723 | 46946 | 31453 | 42% | 2292 | 9224 | 6708 | 11310 | 6328 | 81% |

The results indicate that reproducibility for all four targets was good, with few CVs greater than 60% and many results less than 30%. Consistent results were seen across the range of observed KIs.

### Example 5: mSBA Precision (Repeatability)

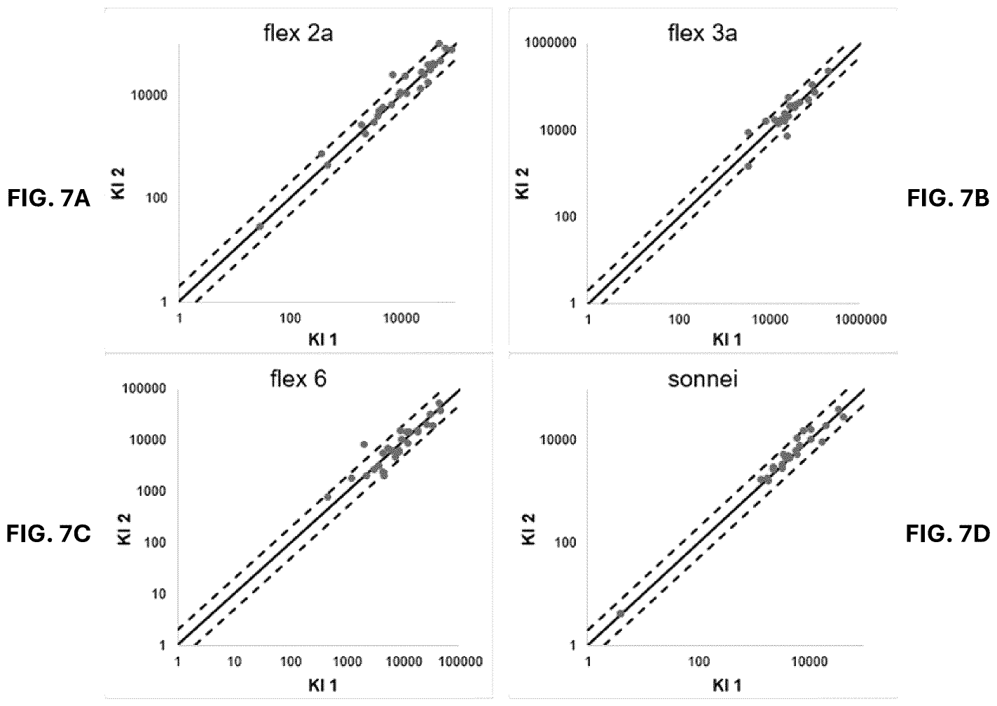

Repeatability of the inventive mSBA assays was also assessed by testing a panel of 30 human sera two times within selected runs. Paired tests where one (or both) result was rejected based on sample acceptance criteria were excluded. Results are shown in Table 3 and in FIG. 7A-D.

Table 3. mSBA Repeatability. Results from repeatability assessments are shown below. The killing indexes (KIs) from the two individual tests per run (KI 1 and KI 2) and the ratio of the two KIs (KI 1/KI 2) are included. KIs below the lower technical limit are indicated as “<TL” and KIs above the technical limit are indicated as “>TL”. NR, no result

| | flex 2a | | | flex 3a | | | flex 6 | | | sonnei | | |
|---|---|---|---|---|---|---|---|---|---|---|---|---|
| | KI 1 | KI 2 | Ratio | KI 1 | KI 2 | Ratio | KI 1 | KI 2 | Ratio | KI 1 | KI 2 | Ratio |
| S01 | 9924 | 10853 | 0.9 | >TL | >TL | NA | 475 | 770 | 0.6 | <TL | <TL | NA |
| S02 | 371 | 706 | 0.5 | 3551 | 8651 | 0.4 | 2012 | 7968 | 0.3 | <TL | NR | NA |
| S03 | 4124 | 4788 | 0.9 | 18057 | 16070 | 1.1 | 9047 | 14838 | 0.6 | 4417 | 4929 | 0.9 |
| S04 | >TL | 9998 | NA | NR | 1915 | NA | >TL | 5294 | NA | <TL | <TL | NA |
| S05 | 40605 | 37914 | 1.1 | 230336 | NR | NA | 33843 | 18297 | 1.8 | 3396 | 2829 | 1.2 |
| S06 | 12179 | 22278 | 0.5 | 32371 | 35479 | 0.9 | 18625 | 15279 | 1.2 | 3362 | 3194 | 1.1 |
| S07 | 29 | 28 | 1.0 | 3411 | 1452 | 2.3 | 128 | NR | NA | NR | <TL | NA |
| S08 | 3436 | 2898 | 1.2 | 74390 | 47189 | 1.6 | 6769 | 5920 | 1.1 | 43427 | 27276 | 1.6 |
| S09 | NR | 6716 | NA | >TL | 6370 | NA | NR | NR | NA | NR | 644 | NA |
| S10 | 488 | 429 | 1.1 | NR | 21709 | NA | 3034 | 2661 | 1.1 | <TL | <TL | NA |
| S11 | 22740 | 12749 | 1.8 | 25572 | 6892 | 3.7 | 8483 | 6019 | 1.4 | NR | 163593 | NA |
| S12 | 1981 | 2625 | 0.8 | 21678 | 23487 | 0.9 | 3780 | 3070 | 1.2 | 35576 | 39972 | 0.9 |
| S13 | 84458 | 71557 | 1.2 | 17121 | 15935 | 1.1 | 1244 | 1741 | 0.7 | 11160 | 15746 | 0.7 |
| S14 | 51355 | 98533 | 0.5 | 37664 | 36554 | 1.0 | 5485 | 6934 | 0.8 | 6059 | 10383 | 0.6 |
| S15 | 9479 | 10012 | 0.9 | 202189 | 216941 | 0.9 | 2218 | 1939 | 1.1 | 4624 | 4442 | 1.0 |
| S16 | 31337 | 36385 | 0.9 | NR | 12233 | NA | 12145 | 8628 | 1.4 | NR | 251611 | NA |
| S17 | 63669 | 76766 | 0.8 | 25760 | 20071 | 1.3 | 4720 | 2011 | 2.3 | 17880 | 9264 | 1.9 |
| S18 | 2393 | 1760 | 1.4 | 22579 | 15115 | 1.5 | 4282 | 2283 | 1.9 | 20579 | 18357 | 1.1 |
| S19 | 51973 | 43023 | 1.2 | 28883 | 35287 | 0.8 | 8458 | 5864 | 1.4 | 6747 | 7657 | 0.9 |
| S20 | 7146 | 6147 | 1.2 | 27009 | 54568 | 0.5 | 43233 | 50790 | 0.9 | 5941 | 6143 | 1.0 |
| S21 | 35878 | 29501 | 1.2 | 35733 | 32715 | 1.1 | 7187 | 4691 | 1.5 | 1861 | 1546 | 1.2 |
| S22 | 26574 | 24047 | 1.1 | 91298 | 103950 | 0.9 | 18266 | 14118 | 1.3 | 6221 | 5031 | 1.2 |
| S23 | 4843 | 5526 | 0.9 | 46221 | 40993 | 1.1 | 31187 | 31185 | 1.0 | 2253 | 2900 | 0.8 |
| S24 | 7448 | 24133 | 0.3 | NR | 40877 | NA | 5808 | 6350 | 0.9 | 7879 | 15317 | 0.5 |
| S25 | 31318 | 16631 | 1.9 | NR | 86937 | NA | 9594 | 10006 | 1.0 | 3492 | 5075 | 0.7 |
| S26 | 4009 | 3900 | 1.0 | 12972 | 17057 | 0.8 | 4306 | 5356 | 0.8 | 1374 | 1637 | 0.8 |
| S27 | 121150 | 90213 | 1.3 | NR | 320380 | NA | 13019 | 14013 | 0.9 | 3848 | 4308 | 0.9 |
| S28 | 38228 | 40065 | 1.0 | 15812 | 13864 | 1.1 | 11540 | 14017 | 0.8 | 2264 | 2613 | 0.9 |
| S29 | 23895 | 26076 | 0.9 | 8431 | 15167 | 0.6 | 26365 | 19763 | 1.3 | 1780 | 1784 | 1.0 |
| S30 | 12853 | 10075 | 1.3 | 97692 | 72550 | 1.3 | 46946 | 36217 | 1.3 | 11310 | 10180 | 1.1 |

Statistics

| | | | | | | | |
|---|---|---|---|---|---|---|---|
| Evaluable pairs | 28 | Evaluable pairs | 21 | Evaluable pairs | 27 | Evaluable pairs | 22 |
| # <2-fold | 27 | # <2-fold | 17 | # <2-fold | 25 | # <2-fold | 22 |
| % <2-fold | 96% | % <2-fold | 81% | % <2-fold | 93% | % <2-fold | 100% |

In the experiments for each target, the KIs for sera tested two times in the same run are shown, with the first KI shown on the X axis and the second on the Y axis. Each graph had a solid black line of identity and dashed black lines indicating 3-fold deviations from identity.

The results in TABLE 3 and FIG. 7A-D indicate that when the inventive mSBA assays are repeated that the results do not substantially vary.

### Example 6: Comparison of mSBA Results Obtained by Different Laboratories

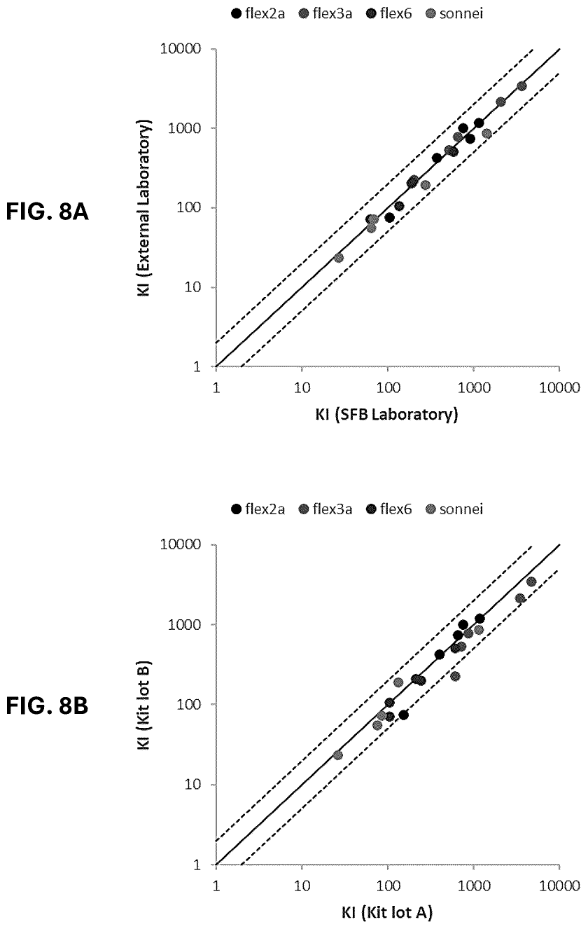

The results of experiments comparing KI values obtained for 4 targets using the inventive SFB mSBA Kit by two different laboratories are in FIG. 8A-B. Five samples were analyzed in the mSBA, including 4 sera and one mixture of monoclonal antibodies specific for each of the 4 target strains. Each graph has a line of identity (solid black line) and lines indicating 2-fold deviations (dashed black lines). (Panel A, Top)

KI values were obtained at a first laboratory (Laboratory A) (x-axis) and a second laboratory (Laboratory B) (y-axis) using the same mSBA Kit lots. (Panel B, Bottom) Two different SFB mSBA kits used in the analysis. The x-axis represents results using mSBA Kit lot A and the y-axis represents results using mSBA Kit lot B.

The results indicate that both laboratories obtained similar KI values against the 4 targets using both lots of SFB mSBA Kit.

### CONCLUSIONS

Based on the performance of the inventive mSBA to date, it appears that the inventive mSBA has substantial potential for usage in vaccine evaluations. The multiplexed format of the assay improves throughput and moreover produces results for four targets, compared to one target with the current singleplex SBAs, with the same volume of test sera—this is particularly important when evaluating samples from infants and children. Also, multiplexing helps to reduce cost by reducing the amounts of expensive reagents (e.g., rabbit complement) needed, as well as reducing labor costs, both of which will help with commercialization of the assay.

The current invention is an improvement of our previous singleplex SBA protocol (Nahm, M. H., et al., Development, Interlaboratory Evaluations, and Application of a Simple, High-Throughput Shigella Serum Bactericidal Assay. mSphere, 2018. 3(3)), which was used to evaluate serum antibody titers for 3 Shigella strains, including S. flexneri 2a, S. flexneri 3a, and S. sonnei, in a singleplex format. Moreover, the further addition of S. flexneri 6 to the SBA extends target strain coverage to 4 serotypes, a combination that aligns with current vaccine development efforts. It has been estimated that an effective multivalent vaccine for the predominant serotypes flex 2a, flex 3a, flex6, and sonnei would provide protection for up to 75% of Shigella strains and potentially higher through cross-protection for non-vaccine strains [13, 17]. Thus, the inventive mSBA format is well suited for the evaluation of putative multivalent Gram negative vaccines, especially multivalent Shigella vaccines.

As was noted in the Background section, mSBA was previously used to evaluate the functionality of anti-cholera antiserum (Yang, J. S., et al., A duplex vibriocidal assay to simultaneously measure bactericidal antibody titers against Vibrio cholerae O1 inaba and Ogawa serotypes. J Microbiol Methods, 2009. 79(3): p. 289-94). However, the mSBA was only two-fold multiplexed and the reduction in serum requirement would not have been significant.

Moreover, laboratory use of antibiotic resistant bacteria can be dangerous. In developing mSBA, we judiciously developed a panel of Shigella target strains that are resistant to one of streptomycin, kanamycin, nalidixic acid, or doxycycline. As each of these antibiotics is not used to treat patients with Shigella infection, this makes the target strains used in our inventive multiplex bactericidal assays safe to handle.

Yet further, the inventive mSBA can be performed in a simplified, robust, economical, and high-throughput manner to determine quantitative killing index (KI) values for approximately 45 samples (analyzed in duplicate) for each of the 4 target strains, thus providing ^{˜}180 KI values per assay. In addition to saving time and resources, the multiplexed assay reduces sample requirements. The reduction in sample need is very important since Shigella vaccines would be used for children who cannot yield high serum volumes. Future improvements, such as using automated liquid handling instruments, have the potential to increase assay throughput without major protocol modifications.

Yet another advantage is that the inventive method requires no special equipment, such as expensive plate readers, that are typically used in certain multiplexed SBA protocols. The Necchi assay, which measures ATP released by the killed bacteria, has a high throughput but requires specialized equipment for ATP measurement (Necchi, F., A. Saul, and S. Rondini, “Development of a high-throughput method to evaluate serum bactericidal activity using bacterial ATP measurement as survival readout”, PLoS One, 2017. 12(2): p. e0172163). Further, the Necchi assay cannot be made into a multiplexed assay.

Further, while multiplexing potentially alternatively could have been achieved using target strains expressing green or red fluorescent protein (GFP vs RFP), this approach would require a dedicated instrument capable of detecting fluorescent markers.

Also, another alternative approach we considered was developing Shigella strains that require a special growth factor or a vitamin, as the creation/use of such strains would not increase biohazard and might be preferred when the target bacteria pose extreme biohazard risk.

During the mSBA development, studies were performed to compare results from singleplex and multiplexed protocols. We obtained similar KI values for all 4 target strains when analyzed by the two methods used side-by-side (data not shown), indicating comparable results from both methods. Accordingly, the use of the inventive mSBA during clinical studies of Shigella or other Gram-negative bacterial vaccines may provide surrogate protection levels.

Yet another advantage of the inventive mSBA methods described herein is that it readily can be adapted to other Gram-negative target bacteria which infect humans such as Salmonella and Meningococci. Vaccines against these bacteria would include multiple serotypes of capsule or LPS, and evaluation of these vaccines would be facilitated by mSBA.

Having described the invention and exemplary embodiments, the invention is further described by the following claims.

What is claimed is:

1. A multiplexed serum-based bactericidal assay (SBA) for detecting the efficacy and/or specificity of a putative vaccine to elicit protective antibodies against 3 or more different gram-negative bacterial strains wherein the bactericidal assay detects the ability of antiserum elicited against the putative vaccine to kill 3 or more different gram-negative bacterial strains optionally of the same genus, by complement mediated-cytotoxicity and wherein the bactericidal assay is conducted in multiplex format using (i) each of said 3 or more gram-negative bacterial strains which respectively are resistant to a different antibiotic relative to the other gram-negative bacterial strains and (ii) complement.

2. The multiplexed SBA of claim 1, which detects the efficacy and/or specificity of a vaccine to elicit protective antibodies against 4 or more different gram-negative bacterial strains of the same genus.

3. The multiplexed SBA of claim 1, wherein said different gram-negative bacterial strains optionally of the same genus are selected from Shigella strains, Salmonella strains, Meningococcal strains; E. coli strains, Klebsiella strains, Pseudomonas strains, Acinetobacter strains, Burkholderia strains, Clostridioides strains, Neisseria strains, Vibrio strains, Serratia strains, Campylobacter strains, Bordatella strains, Yersinia strains, Legionella strains, Haemophilus influenzae strains, Pasteurella multocida strains, Enterobacter strains, Bacteroides fragilis strains, Proteus strains, and Helicobacter pylori strains.

4. The multiplexed SBA of claim 3, wherein said different gram-negative bacterial strains comprise at least 3 or all 4 of S. flexneri 2a, S. flexneri 3a, S. sonnei, and S. flexneri 6.

5. The multiplexed SBA of claim 4, wherein the S. flexneri 2a is resistant to streptomycin and sensitive to the other three antibiotics, the S. flexneri 6 strain is resistant to nalidixic acid and sensitive to the other three antibiotics, the S. flexneri 3a strain is resistant to kanamycin and sensitive to the other three antibiotics, and the S. sonnei is resistant to doxycycline and sensitive to the other three antibiotics.

6. The multiplexed SBA of claim 1, wherein each of the target bacterial strains is resistant to the antibiotic used for selection but sensitive to the other antibiotic used for selection of the other target strains used in the multiplexed bactericidal assay.

7. The multiplexed SBA of claim 1, wherein each of the antibiotic resistant strains used in the assay is resistant to an antibiotic generally not used for treatment against the target bacterial strain.

8. The multiplexed SBA of claim 1, wherein the growth of each target strain is not substantially affected by the resistant antibiotic at twice the working concentration used in the bactericidal assay, but growth is completely inhibited by the sensitive antibiotics at one-half the working concentrations used in the bactericidal assay.

9. The multiplexed SBA of claim 1, wherein the antiserum is obtained from a subject immunized with a putative multivalent vaccine developed to protect against the at least 4 different gram-negative bacterial strains.

10. The multiplexed SBA of claim 1, wherein the antiserum is obtained from an infant or child immunized with a putative multivalent vaccine developed to protect against the different gram-negative bacterial strains.

11. The multiplexed SBA of claim 1, wherein said putative vaccine contains different bacterial antigens, optionally glycoproteins, further optionally O-antigens derived from the different target bacterial strains.

12. The multiplexed SBA of claim 1, wherein said putative vaccine contains an antigen, optionally a glycoprotein, further optionally an O-antigen or fragment thereof which elicits protective or neutralizing antibodies against 2 or more of the target bacterial strains.

13. The multiplexed SBA of claim 1, wherein said putative vaccine contains a dominant epitope or consensus antigen, optionally a glycoprotein, further optionally an O-antigen or fragment thereof which elicits protective or neutralizing antibodies against 2 or more of the target bacterial strains.

14. The multiplexed SBA of claim 1, which includes the use of human or rabbit complement, optionally baby rabbit complement (BRC).

15. The multiplexed SBA of claim 1, which is used to evaluate the efficacy and/or specificity of a vaccine using antiserum obtained from a vaccinated subject after at least one administration of the vaccine.

16. The multiplexed SBA of claim 1, which is used to evaluate the efficacy and/or specificity of using antiserum obtained from a vaccinated subject after multiple administrations of the vaccine, optionally wherein said multiple administrations are effected at different times.

17. The multiplexed SBA of claim 1, which is used to evaluate the duration of efficacy and/or specificity of a vaccine using antiserum obtained from a vaccinated subject after a period of at least 1 week, 2 weeks, 3 weeks, 1 month, 2 months, 3 months, 4 months, 5 months, 6 months, a year or longer after the last administered dosage of the vaccine.

18. The multiplexed SBA of claim 1, which is used to evaluate the optimal dosing interval and/or dosage of the vaccine using antiserum obtained from one or more vaccinated subjects.

19. A kit for performing a multiplexed SBA according to claim 1 which comprises 3 or more frozen or lyophilized target bacteria which are respectively resistant to different antibiotics and complement, optionally rabbit complement and instructions for using the kit.

20. The kit of claim 19, which additionally comprises other reagents such as buffers and antibiotics.

21. The kit claim 19, which comprises one, two, three or all four of streptomycin, nalidixic acid, kanamycin and doxycycline.
